(19) United States
(12) Patent Application Publication
(10) Pub. No.: US 2016/0297137 A1
(43) Pub. Date: Oct. 13, 2016

(54) 3D EMBOSSING TECHNOLOGY FOR NONWOVENS

(71) Applicant: First Quality Nonwovens, Inc., Great Neck, NY (US)

(72) Inventors: Peter ZAJACZKOWSKI, Media, PA (US); John C. PARSONS, Dallas, PA (US); James SEALEY, Belton, SC (US); Justin PENCE, Anderson, SC (US)

(21) Appl. No.: 15/096,000

(22) Filed: Apr. 11, 2016

Related U.S. Application Data
(60) Provisional application No. 62/145,576, filed on Apr. 10, 2015.

(51) Int. Cl. B29C 59/04; A61F 13/15; B29C 59/00

(52) U.S. Cl. CPC B29C 59/04; B29C 59/002; A61F 13/15731; B29K 2023/06; B29K 2023/12

(57) ABSTRACT

A method of manufacturing an embossed nonwoven sheet comprising feeding a sheet comprising spunmelt fibers through a nip region of a forming station, the forming station comprising a rigid cylinder having a surface with raised elements that form a 3D pattern and a deformable opposing surface which is not part of the rigid cylinder, so that the sheet is in contact with both the rigid cylinder and the deformable opposing surface at the nip region as the sheet moves through the forming station and the 3D pattern is embossed onto the sheet.

### RELATED APPLICATION

This application is a non-provisional based on and claiming priority to U.S. Provisional Patent Application No. 62/145,576, filed Apr. 10, 2015, the contents of which are incorporated herein by reference in their entirety.

### FIELD OF THE INVENTION

The present invention is directed to embossing of sheets of material, and in particular, to 3D embossing of nonwoven material.

### BACKGROUND OF THE INVENTION

Recently there has been a significant change in the types of nonwovens being used in premium hygiene products, most notably baby diapers. The materials being used have transitioned from those having a relatively 2D structure to those with higher bulk and in many cases with a pronounced visible 3D pattern. The bulk of these structures has increased from around 200 microns for the relatively “flat” 2D materials to approaching 1000 microns for the 3D pattern, and in some cases exceeding that.

These new structures can offer multiple advantages to diaper producers and their consumers such as:

1) a visible 3D pattern conveys a high quality image and can lead to a perception of improved performance;
2) a higher bulk structure provides greater separation between the skin-contacting surface and the absorbent core. In the case of diapers, this keeps any body liquids further away from the skin and can lead to a drier diaper;
3) a high bulk 3D topsheet can allow the use of a thinner, lower cost, acquisition layer; and
4) a higher bulk structure can also provide a more open structure to quickly absorb runny liquids and prevent leakage or reduce skin irritation.

In addition, with the trend towards thinner, higher SAP cores there is a need for additional cushioning between the (typically) stiffer cores and the outer layers of a diaper.

Prior to the introduction of these new materials, the majority of the nonwovens used in baby diapers were produced using spunmelt processes to form and draw continuous filaments and create a nonwoven comprising randomly oriented filaments. The spunmelt process is capable of producing soft and strong materials at high speed and low cost. However, the new materials do not lend themselves to the typical spunmelt process because it is difficult to impart bulk and 3D patterning. In addition, the new materials work better if they have a resilient structure so that the bulk and 3D pattern remain in place during roll winding, storage, and compression packaging of the finished product.

In order to produce the new materials manufacturers had reverted to older nonwoven processes, such as carding and through-air bonding. In the carding process individual fibers (staple) are combed to orient them in the machine direction (MD) and to produce a uniform unbonded web. This web is then passed through a through-air oven (typically a long MD tunnel or a drum type oven) where the fibers are heated and bonded together. The fibers usually contain a low melt component that acts as the bonding agent while the higher melt component maintains the structural integrity of the web. The advantage to this process is that it readily allows for the inclusion of different fiber and polymer types so that it is easy to add, e.g., polyester fiber to provide resilience. The staple fibers come with a crimp so they naturally provide more bulk. The general slower line speeds (below 500 m/min, typically ˜200-300 m/min) also allow more time for patterns to be included.

However, the use of these materials, combined with the slower production processes and higher cost raw material (staple vs. resin) is undesirable for diaper producers and there is a demand for lower cost materials that offer the same performance. In short, while current spunmelt processes are very cost effective, they do not currently work well in generating high loft 3D structures.

The present invention relates to processes and apparatus for producing 3D patterns, including high loft 3D patterns, in high speed spunbond processes.

### SUMMARY OF THE INVENTION

This invention relates to a 3D embossing technology which enables production of 3D patterns on spunmelt sheets at high speeds using a configuration that permits an extended dwell time in a forming section and longer nip lengths. High loft 3D patterns can be obtained at relatively high speeds. In addition, the production apparatus is cheaper to produce and quicker to make than traditional calender systems.

A process is provided for producing a 3D pattern on a spunmelt sheet comprising feeding the spunmelt sheet through a forming station, the forming station comprising a rigid cylinder having a surface which is pressed against a deformable opposing surface which is not part of the rigid cylinder, wherein the surface of the rigid cylinder has a 3D pattern thereon, and wherein the spunmelt sheet is moved between the surface of the rigid cylinder and the deformable opposing surface, and thereby pressed onto the 3D pattern of the rigid cylinder so as to emboss a 3D pattern on the spunmelt sheet.

Also provided is a process for producing a 3D pattern on a spunmelt sheet comprising feeding the spunmelt sheet through a forming station, the forming station comprising (i) a rigid cylinder having a surface comprising holes in the surface thereof permitting suction pressure to be applied to the surface of the spunmelt sheet in contact with the rigid cylinder, the rigid cylinder positioned close enough to (ii) an opposing surface which is not part of the rigid cylinder, so as to permit a spunmelt sheet which on one surface thereof is in contact with the surface of the rigid cylinder to also be in contact on a second surface of the spunmelt sheet with the opposing surface, wherein the surface of the rigid cylinder has a 3D pattern thereon, and wherein the spunmelt sheet is moved between the surface of the rigid cylinder and the opposing surface and suction pressure is applied such that the spunmelt sheet is thereby pressed onto the 3D pattern of the rigid cylinder so as to emboss a 3D pattern on the spunmelt sheet.

Also provided is an apparatus for producing a 3D pattern on a spunmelt sheet, the apparatus comprising a forming station, the forming station comprising a rigid cylinder having a surface which is pressed against a deformable opposing surface which is not part of the rigid cylinder, wherein the surface of the rigid cylinder has a 3D pattern thereon, and wherein the spunmelt sheet is moved between the surface of the rigid cylinder and the deformable opposing surface and thereby pressed onto the 3D pattern of the rigid cylinder so as to emboss a 3D pattern on the spunmelt sheet.

Also provided is an apparatus for producing a 3D pattern on a spunmelt sheet, the apparatus comprising a forming station, the forming station comprising (i) a rigid cylinder having a surface comprising holes in the surface thereof permitting suction pressure to be applied to the surface of the spunmelt sheet in contact with the rigid cylinder, the rigid cylinder positioned close enough to (ii) an opposing surface which is not part of the rigid cylinder, so as to permit a spunmelt sheet which on one surface thereof is in contact with the surface of the rigid cylinder to also be in contact on a second surface of the spunmelt sheet with the opposing surface, wherein the surface of the rigid cylinder has a 3D pattern thereon, and wherein the spunmelt sheet is moved between the surface of the rigid cylinder and the opposing surface and suction pressure is applied such that the spunmelt sheet is thereby pressed onto the 3D pattern of the rigid cylinder so as to emboss a 3D pattern on the spunmelt sheet.

### BRIEF DESCRIPTION OF FIGURES

FIG. 1 is a side, schematic, cross-sectional view of an apparatus wherein a spunmelt sheet is pressed between a deformable wire and a patterned cylinder according to an exemplary embodiment of the present invention.

FIG. 2 is a side, schematic, cross-sectional view of an apparatus wherein a spunmelt sheet is pressed between a deformable wire and a patterned cylinder according to an exemplary embodiment of the present invention.

### DETAILED DESCRIPTION OF THE INVENTION

Current high speed spunmelt lines run at >500 m/min, especially for producing lightweight nonwovens. The fibers are produced by melting resin pellets, extruding them onto a porous belt and then tacking them down under a heated press roll. The resulting web is then typically bonded using a thermal calender. The calender process typically uses two large steel heated rolls (in contact under high pressure) and the web is fed through the nip created between the rolls. One roll is usually smooth while the other roll has a raised pattern. The heat and pressure exerted at the nip melts the polymer in the fiber at the raised portions and creates a bonding pattern that provides the structural integrity of the web.

The typical nip distance in machine direction (MD) is quite small (usually no more than a few millimetres) so that the dwell time of the web in the nip is measured in microseconds. This is insufficient time for the filaments of the fabric to soften and form around a pronounced 3D pattern. In addition the high pressures used in the current calender designs means that the raised patterns cannot be very high (usually less than 1 mm) because a higher raised bond figure (point) is prone to fracture.

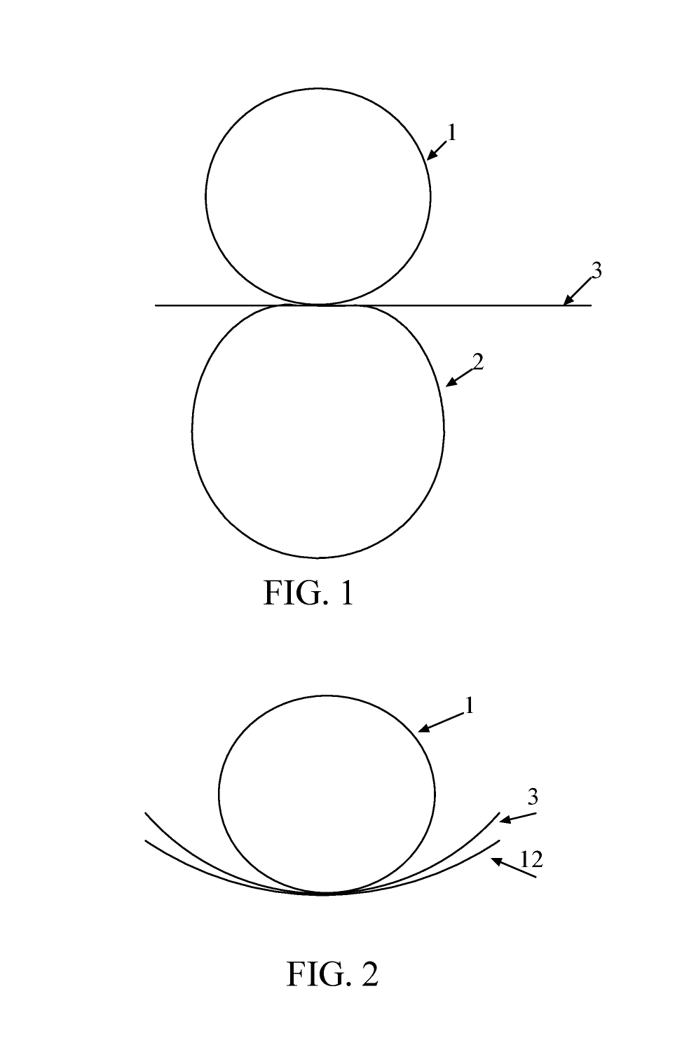

Herein a process is disclosed wherein in order to create a pronounced 3D structure from the spunmelt process, the dwell time of the fabric within the “forming” section is increased. A “wire” or flexible belt can be used in the patterning mechanism. In a shoe press configuration embodiment, (FIG. 1), the flexible wire can be in a cylindrical form on the bottom roll. The top cylinder “pushes” into the wire on the bottom cylinder thus creating a much longer path for the spunmelt sheet to be embossed.

The sizes of the cylinders and the flexibility of the wire/belt can be adjusted to deliver preferred nip lengths and dwell times. The flexible wire or belt can be produced with high melting point polymers so that they do not soften under the bonding temperatures needed for the typical thermoplastics (PP & PE) used in spunmelt fabrics.

In another embodiment the cylinder area before the nip section is employed as a pre-heating mechanism to increase the ability to form the material in a shorter amount of time. Also, if desired, multiple “pushing” cylinders can be positioned around the flexible wire cylinder so that forming and bonding can be effected in separate stages. Alternate press designs used in tissue making may also be used to provide longer nip lengths and dwell times.

A “wire” used to provide 3D bonding with spunmelts as described herein has significant advantages in time and cost over traditional calender systems. Traditional calender rolls can cost >$500K and can have lead times approaching one year. In contrast, a flexible molded belt is much quicker and cheaper to produce and would allow, for example, diaper manufacturers using the process to change patterns more frequently.

A process is provided for producing a 3D pattern on a spunmelt sheet comprising feeding the spunmelt sheet through a forming station, the forming station comprising a rigid cylinder having a surface which is pressed against a deformable opposing surface which is not part of the rigid cylinder, wherein the surface of the rigid cylinder has a 3D pattern thereon, and wherein the spunmelt sheet is moved between the surface of the rigid cylinder and the deformable opposing surface, and thereby pressed onto the 3D pattern of the rigid cylinder so as to emboss a 3D pattern on the spunmelt sheet.

The deformable opposing surface can be on a wire or a belt pressing on the rigid cylinder through the spunmelt sheet. The deformable opposing surface can be a deformable surface on a wire in cylindrical form.

The spunmelt sheet may be moved through the forming station at least in part by being held against the rigid cylinder by the wire or a belt pressing the spunmelt sheet against the rigid cylinder, and by the rotation of the rigid cylinder and/or the wire or belt.

The surface of the rigid cylinder can comprise thereon a 3D pattern. In an embodiment, the deformable opposing surface also comprises thereon a 3D pattern. The deformable opposing surface can also not have a 3D pattern thereon.

In an embodiment, the 3D pattern is from 0.1 mm to 5 mm in depth and may be, for example, from 0.1 mm to 3 mm in depth, from 0.1 mm to 2 mm in depth, from 0.1 mm to 1 mm in depth, or from 0.1 mm to 0.5 mm in depth.

The spunmelt sheet may be produced from a resin. The spunmelt sheet can, optionally, be heated prior to contact with the rigid cylinder to assist embossing of the 3D pattern on the spunmelt sheet. If desired, at least a portion of the rigid cylinder in contact with the spunmelt sheet is heated so as to assist embossing of the 3D pattern on the spunmelt sheet. Also, contact points on the 3D pattern on the rigid cylinder can have an adhesive applied thereto if the user desires.

In a preferred embodiment of the invention, the spunmelt sheet includes one or more layers of substantially continuous fibers or filaments and is a spunbond, meltblown and/or spunbond-meltblown-spunbond (“SMS”) web. In embodiments of the invention, the spunmelt sheet can be made from mono component, bi-component, or multi-component fibers.

In embodiments, the deformable opposing surface is a deformable surface on a plastic mesh wire in cylindrical form. The deformable opposing surface may comprise a rubber, a woven fabric, a cased film, an extruded film, or an overlaid fabric with polymer elements applied to the surface thereof, a plastic, or nested steel.

Optionally, the deformable opposing surface is heated so as to assist embossing of the 3D pattern on the spunmelt sheet.

The process can produce a spunmelt sheet having a 3D pattern wherein the 3D pattern from 100 microns to in excess of 1.0 mm peak to trough. For example, the process can produce a spunmelt sheet having a 3D pattern wherein the 3D pattern is from 400 microns to 3100 microns from peak to trough, or from 500 microns to 3000 microns from peak to trough.

The rigid cylinder can comprise steel. The 3D pattern on the rigid cylinder may comprise steel.

The process can comprise feeding the spunmelt sheet through a nip, wherein the nip is of a belt press, a shoe press, a visco nip press, a double-nip press or a multi-nip press. In an embodiment, the spunmelt sheet is fed through a nip of an ATMOS press.

The nip can be a belt press which is porous to liquid. The nip can be a belt press that is non-porous to liquid. Said belt presses can be steam-heated.

The spunmelt sheet can be fed through the forming station at a high speed, such as a speed in excess of 400 m/min. In an embodiment, the spunmelt sheet is fed through the forming station at a speed in excess of 500 m/min.

The rigid cylinder may comprise holes in the surface thereof permitting suction pressure to be applied to the surface of the spunmelt sheet in contact with the rigid cylinder so as to hold the spunmelt sheet on the rigid cylinder.

Steam heat may be applied to the rigid cylinder and/or to the spunmelt sheet in contact therewith. In an embodiment, steam heat is applied via a steam hood.

In an embodiment, the flexible opposing surface is made from a plastic that has a melting point higher than the melting point of polypropylene or has a melting point higher than the melting point of polyethylene.

Also provided is a process for producing a 3D pattern on a spunmelt sheet comprising feeding the spunmelt sheet through a forming station, the forming station comprising (i) a rigid cylinder having a surface comprising holes in the surface thereof permitting suction pressure to be applied to the surface of the spunmelt sheet in contact with the rigid cylinder, the rigid cylinder positioned close enough to (ii) an opposing surface which is not part of the rigid cylinder, so as to permit a spunmelt sheet which on one surface thereof is in contact with the surface of the rigid cylinder to also be in contact on a second surface of the spunmelt sheet with the opposing surface, wherein the surface of the rigid cylinder has a 3D pattern thereon, and wherein the spunmelt sheet is moved between the surface of the rigid cylinder and the opposing surface and suction pressure is applied such that the spunmelt sheet is thereby pressed onto the 3D pattern of the rigid cylinder so as to emboss a 3D pattern on the spunmelt sheet.

Steam heat can be applied to the rigid cylinder and/or to the spunmelt sheet in contact therewith. The steam heat can be applied via a steam hood.

The opposing surface can be a second cylinder. The opposing surface can comprise rubber and/or steel.

Also provided is an apparatus for producing a 3D pattern on a spunmelt sheet comprising a forming station, the forming station comprising a rigid cylinder having a surface which is pressed against a deformable opposing surface which is not part of the rigid cylinder, wherein the surface of the rigid cylinder has a 3D pattern thereon, and wherein the spunmelt sheet is moved between the surface of the rigid cylinder and the deformable opposing surface and thereby pressed onto the 3D pattern of the rigid cylinder so as to emboss a 3D pattern on the spunmelt sheet.

Also provided is an apparatus for producing a 3D pattern on a spunmelt sheet comprising a forming station, the forming station comprising (i) a rigid cylinder having a surface comprising holes in the surface thereof permitting suction pressure to be applied to the surface of the spunmelt sheet in contact with the rigid cylinder, the rigid cylinder positioned close enough to (ii) an opposing surface which is not part of the rigid cylinder so as to permit a spunmelt sheet which on one surface thereof is in contact with the surface of the rigid cylinder to also be in contact on a second surface of the spunmelt sheet with the opposing surface, wherein the surface of the rigid cylinder has a 3D pattern thereon, and wherein the spunmelt sheet is moved between the surface of the rigid cylinder and the opposing surface and suction pressure is applied such that the spunmelt sheet is thereby pressed onto the 3D pattern of the rigid cylinder so as to emboss a 3D pattern on the spunmelt sheet.

In an embodiment of the process or apparatus, a steel patterned emboss roll to rubber roll is employed with heating. The heat can be applied to the sheet pre-emboss (such that the emboss points would not need to be heated). In an embodiment, the heat can be applied on the steel roll.

In an embodiment of the process or apparatus, a steel patterned emboss roll can be employed with adhesive applied to points. In tissue manufacturing, a glue is commonly used to set/hold the embossed pattern in a one-ply or two-ply structure. Glue may also be used to bond two or more plies together to generate thickness for a multi-ply structure. Heat is typically required to set the glue. The glue is often applied in a pattern by applying it only to the raised points of an embossing roll. In an embodiment, the adhesive can be applied after emboss and rubber roll to the top of emboss point by spray application (e.g. inside of pocket) or roll “kiss” applications (emboss top side, outer). Heat can be applied to the sheet before embossing. Cooling can be effected after application of the bonding agent.

In an embodiment of the process or apparatus, the emboss pattern can be applied at the product production plant (e.g. a diaper-making facility) with a compression roll that is heated and textured.

The emboss station can employ steel to rubber, nested-steel to steel, or tip to tip rollers or belts. In tip to tip two embossing rolls are positioned with the raised portions thereof, or tips, matching. This can provide greater pattern depth because the distance from one “trough” to the next (on the opposing roll) is twice what one would achieve with just one patterned roll. This would be unorthodox for use in nonwovens because the pressures otherwise required generally make the system not very robust.

In an embodiment of the process or apparatus, the wire is a woven fabric, is cased, is extruded film, or is overlaid (e.g. fabric with polymer elements applied to surface)

In both the process and apparatus, a suction roll 3D emboss section can be used with steam heating (steam hood). The 3D emboss pattern can be controlled by texture of a suction roll surface. A rubber roll or a steel roll are options for use on the bottom side of the suction roll to produce a nip.

An apparatus in which the new technology can be employed in a shoe press configuration for producing 3D patterns according to an exemplary embodiment of the present invention is shown in FIG. 1. The flexible wire can be in a cylindrical form on the bottom roll 2. The top cylinder 1 “pushes” into the wire on the bottom roll 2 thus creating a much longer nip region along the perimeter of the top cylinder and a correspondingly longer dwell time during which nonwoven sheet 3 is pressed between both the flexible wire 2 and the top cylinder 1. Another apparatus in which the 3D embossing process can be employed according to an exemplary embodiment of the present invention is shown in FIG. 2, where a deformable belt 12 presses the nonwoven 3 web against a patterned top cylinder 1, resulting in an extended nip region and a longer dwell time.

In exemplary embodiments of the invention, the nip regions may extend at least 2 cm around the perimeter of the patterned cylinder 1. Preferably, the nip regions extend at least 4 cm around the perimeter of the patterned cylinder 1. In some embodiments the nip regions can extend at least 10 cm around the perimeter of the patterned cylinder 1. An advantage of the present invention is that the longer nip regions allow for an increased machine speed while still providing a sufficient dwell time for embossing.

Also provided are products made using the 3D embossed spunmelt sheets produced as described herein. Such products encompassed include personal hygiene products. In an embodiment, the product is a personal hygiene product for holding menses. In an embodiment, the product is a diaper for holding urine and/or fecal matter.

“And/or” as used herein, for example, with option A and/or option B, encompasses the separate embodiments of (i) option A, (ii) option B, and (iii) option A plus option B.

Where a numerical range is provided herein, it is understood that all numerical subsets of that range, and all the individual integers contained therein, are provided as part of the invention. Thus, a structure for which the range provided is from 500 to 3000 microns peak to trough includes the inventions of the subset of structures which are 500 to 2000 microns peak to trough, the subset of primers which are 600 to 1000 peak to trough etc., as well as a structure which is 750 microns peak to trough, a structure which is 1000 microns peak to trough, a structure which is 1500 microns peak to trough, etc. up to and including a structure which is 3000 microns peak to trough.

All combinations of the various elements described herein are within the scope of the invention unless otherwise indicated herein or otherwise clearly contradicted by context.

1. A method of manufacturing an embossed nonwoven sheet comprising the steps of:
feeding a sheet comprising spunmelt fibers through a nip region of a forming station, the forming station comprising a rigid cylinder having a surface with raised elements that form a 3D pattern and a deformable opposing surface which is not part of the rigid cylinder, so that the sheet is in contact with both the rigid cylinder and the deformable opposing surface at the nip region as the sheet moves through the forming station and the 3D pattern is embossed onto the sheet.

2. The method of claim 1 further comprising the step of applying suction pressure to press the sheet against the surface of the rigid cylinder.

3. The method of claim 1 wherein the deformable opposing surface is a belt and the nip region extends at least 2 cm around the perimeter of the rigid cylinder.

4. The method of claim 1 wherein the deformable opposing surface is a belt and the nip region extends at least 4 cm around the perimeter of the rigid cylinder.

5. The method of claim 1 wherein the deformable opposing surface is a belt and the nip region extends at least 10 cm around the perimeter of the rigid cylinder.

6. The method of claim 1 wherein the deformable opposing surface is a flexible cylinder and the nip region extends at least 2 cm around the perimeter of the rigid cylinder.

7. The method of claim 1 wherein the deformable opposing surface is a flexible cylinder and the nip region extends at least 4 cm around the perimeter of the rigid cylinder.

8. The method of claim 1 wherein the deformable opposing surface is a flexible cylinder and the nip region extends at least 10 cm around the perimeter of the rigid cylinder.

9. The method of claim 1 wherein the deformable opposing surface comprises a rubber surface and the nip region extends at least 2 cm around the perimeter of the rigid cylinder.

10. The method of claim 1 further comprising the step of heating the nip region using steam provided by a steam hood.

11. An apparatus for embossing a nonwoven sheet comprising:
a rigid cylinder comprising a perimeter surface with a pattern of protrusions;
a flexible cylinder comprising a deformable perimeter surface opposed to the perimeter surface of the rigid cylinder; and
a nip region located between the perimeter surface of rigid cylinder and the deformable perimeter surface of the flexible cylinder,
wherein the nip region extends at least 2 cm along the perimeter surface of the rigid cylinder.

12. The apparatus of claim 11 wherein the flexible cylinder comprises a plastic mesh wire.

13. An apparatus for embossing a nonwoven sheet comprising;
a rigid cylinder comprising a perimeter surface with a pattern of protrusions;
a belt comprising a deformable surface opposed to the perimeter surface of the rigid cylinder;
a nip region located between the perimeter surface of the rigid cylinder and the deformable surface of the belt;
wherein the nip region extends at least 2 cm along the perimeter surface of the rigid cylinder.

14. The apparatus of claim 13 further comprising a steam hood.
